(19) United States
(12) Patent Application Publication
(10) Pub. No.: US 2024/0209366 A1
(43) Pub. Date: Jun. 27, 2024

(54) HIGH EFFICIENCY TRANS-SPLICING FOR REPLACEMENT OF TARGETED RNA SEQUENCES IN HUMAN CELLS

(71) Applicant: Tacit Therapeutics, Inc., South San Francisco, CA (US)

(72) Inventor: David Allen Nelles, San Francisco, CA (US)

(21) Appl. No.: 18/485,535

(22) Filed: Oct. 12, 2023

Related U.S. Application Data
(63) Continuation of application No. PCT/US2022/020151, filed on Mar. 14, 2022.
(60) Provisional application No. 63/175,223, filed on Apr. 15, 2021.

(51) Int. Cl. C12N 15/113; C12N 15/63

(52) U.S. Cl. CPC C12N 15/113; C12N 15/63; C12N 2310/11; C12N 2320/33; C12N 2830/48

(57) ABSTRACT

Disclosed are compositions comprising: a trans-splicing nucleic acid comprising (a) one or more replacement domains that encode a therapeutic sequence operably linked to; (b) one or more intronic domains that promote RNA splicing of the replacement domain comprising a trans-splicing enhancer sequence; and (c) one or more antisense domains that promote binding to a target RNA molecule, wherein an RNA trans-splicing reaction promotes highly efficient insertion of the replacement domain into a target RNA. Methods of making and methods of using compositions of the disclosure are also provided, including but not limited to compositions of the disclosure that may be used in the treatment of a disease or disorder in a patient or subject. Example disease or disorders of the disclosure include genetic and epigenetic diseases or disorders.

### CROSS-REFERENCE

This application is a continuation of International Application No. PCT/US2022/020151, filed Mar. 14, 2022, which claims the benefit of U.S. Provisional Application No. 63/175,223, filed Apr. 15, 2021, each of which application is incorporated herein by reference.

### FIELD OF THE DISCLOSURE

The present disclosure is directed, generally, to gene therapy, molecular biology, and compositions and methods for altering the sequence composition of RNA molecules.

### BACKGROUND

There is a long-felt but unmet need in the art for replacing disease-causing RNA sequences. The disclosure provides compositions and methods for replacement of specific RNA sequences in RNA molecules in human cells with high efficiency. Specifically, the present disclosure provides compositions and methods for replacement of chosen RNA sequences within target RNAs using RNA trans-splicing molecules that carry sequences that increase trans-splicing efficiency to treat a disease in the context of a human gene therapy

Effective treatment of human genetic disease necessitates efficient replacement of defective genetic sequences in human cells. RNA trans-splicing has been proposed as a human gene therapeutic but has not experienced success in clinical trials due to low efficiency. The present disclosure describes improvements to RNA trans-splicing molecules that could address this long-felt but unmet need.

### SUMMARY

The present disclosure provides, in some embodiments, a composition comprising a trans-splicing RNA molecule comprising (a) at least one domain that promotes trans-splicing (“Intronic Domain”) that comprises one or more trans-splicing enhancer sequences, (b) at least one binding domain (“Antisense Domain”) that contains or consists of a sequence complementary to a pre-mRNA present in a human cells (“Target RNA”), and (c) a coding domain that is inserted into the Target RNA via trans-splicing (“Replacement Domain”). The trans-splicing enhancer sequences increase splicing efficiency so that the trans-splicing RNA molecule can exchange sequences within the Target RNA with the Replacement Domain with high efficiency. In other embodiments, the present disclosure provides a composition comprising a nucleic acid sequence encoding the trans-splicing RNA molecule.

In some embodiments, the trans-splicing enhancer sequences comprise 5′-X₁X₂X₃X₄X₅X₆-3′ wherein X₁ is uracil (U) or guanine (G); X₂ is adenine (A), uracil (U) or guanine (G); X₃ is adenine (A), uracil (U) and guanine (G); X₄ is adenine (A), uracil (U), cytosine (C) or guanine (G); X₅ is adenine (A), cytosine (C), uracil (U) or guanine (G); and X₆ is adenine (A), uracil (U) or guanine (G).

In some embodiments, the trans-splicing enhancer sequences comprise X₁X₂X₃X₄X₅X₆ wherein; X₁ is selected from the group including adenine (A), uracil (U) and guanine (G); X₂ is selected from the group including adenine (A), uracil (U) and guanine (G); X₃ is selected from the group including adenine (A), uracil (U) and guanine (G); X₄ is selected from the group including adenine (A), uracil (U) and guanine (G); X₅ is selected from the group including adenine (A), uracil (U) and guanine (G); and X₆ is selected from the group including uracil (U) and guanine (G).

In some embodiments, the trans-splicing enhancer sequences comprise X₁X₂X₃X₄X₅X₆ wherein; X₁ is selected from the group including adenine (A), uracil (U) and guanine (G); X₂ is selected from the group including uracil (U) and guanine (G); X₃ is selected from the group including adenine (A), uracil (U) and guanine (G); X₄ is selected from the group including uracil (U) and guanine (G); X₅ is selected from the group including uracil (U) and guanine (G); and X₆ is selected from the group including uracil (U) and guanine (G).

In some embodiments, the trans-splicing enhancer sequences are directly adjacent to the Replacement domain.

In some embodiments, the trans-splicing enhancer sequences are 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 400 nucleotides, 500 nucleotides, more than 500 nucleotides, or any number of nucleotides in between distant from the first nucleotide of the Replacement Domain in the 5′ direction.

In some embodiments, the trans-splicing enhancer sequences are 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 400 nucleotides, 500 nucleotides, more than 500 nucleotides, or any number of nucleotides in between distant from the last nucleotide of the Replacement Domain in the 3′ direction.

In some embodiments, the Intronic Domain comprises 1 trans-splicing enhancer sequence. In some embodiments, the Intronic Domain comprises 2 or more trans-splicing enhancer sequences. In some embodiments, the Intronic Domain comprises 3, 4, 5, 6, 7, 8, 9, 10, 11, 12, 13, 14, 15, 20, 30, 40, 50, 75, 100, 200, 300 or more trans-splicing enhancer sequences.

In some embodiments, the Antisense Domain is complementary to a gene (corresponding accession numbers in brackets, corresponding disease in parentheses) that encodes a Target RNA that sometimes carry disease-causing mutations and is selected from the group consisting of: TNFRSF13B [ENSG00000240505] (common variable immune deficiency); ADA, CECR1 [ENSG00000196839, ENSG00000093072] (Adenosine deaminase deficiency); IL2RG [ENSG00000147168] (X-linked severe combined immunodeficiency); HBB [ENSG00000244734] (Beta-thassalemia); HBA1, HBA2 [ENSG00000206172, ENSG00000188536] (alpha-thassalemia); U2AF1 [ENSG00000160201] (myelodysplastic syndrome); SOD1, TARDBP, FUS, MATR3, SOD1, C9ORF72 [ENSG00000142168, ENSG00000120948, ENSG00000089280, ENSG00000015479, ENSG00000142168, ENSG00000147894] (Amyotrophic lateral sclerosis); MAPT, PGRN [ENSG00000186868, ENSG00000030582] (Frontotemporal dementia with parkinsonism); CDH23, MYO7A, USH2A [ENSG00000107736, ENSG00000137474, ENSG00000042781] (Usher's syndrome); GALC [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINK1, ATPGAP2 [ENSG00000158828] (early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995,ENSG00000112592, ENSG00000102057, ENSG00000102466] (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646] (genetic epilepsy disorders); ATM [ENSG00000149311] (Ataxia-telangiectasia); GLB1 [ENSG00000170266] (GM1 gangliosidosis); GBA [ENSG00000177628] (Gaucher disease); GM2A [ENSG00000196743] (GM2 gangliosidosis); UBE3A [ENSG00000114062] (Angelman syndrome); SLC2A1 [ENSG00000117394] (glucose transporter deficiency type 1); LAMP2 [ENSG00000005893] (Danon disease); GLA [ENSG00000102393] (Fabry disease); PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG00000130203] (Familial hypercholesterolemia); MYOC, OPTN, TBK1, WDR36, CYPIB1 [ENSG00000034971, ENSG00000123240, ENSG00000183735, ENSG00000134987, ENSG00000138061] (Open Angle Glaucoma); IDUA [ENSG00000127415] (Hurler syndrome or Mucopolysaccharidosis 1); IDS [ENSG00000010404] (Hunter syndrome or Mucopolysaccharidosis 2); CLN3 [ENSG00000188603] (Batten disease); DMD [ENSG00000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SGCB [ENSG00000163069] (Limb-girdle muscular dystrophy type 2E); SGCG [ENSG00000102683] (Limb-girdle muscular dystrophy type 2C); SGCD [ENSG00000170624] (Limb-girdle muscular dystrophy type 2F); DUX4 [ENSG00000260596] (Facioscapulohumeral muscular dystrophy); F9 [ENSG00000101981] (Hemophilia B); F8 [ENSG00000185010] (Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DFNA1, WFS1 [ENSG00000165474, ENSG00000121742, ENSG00000242866, ENSG00000131504, ENSG00000109501] (autosomal dominant hearing impairment); POU3F3 [ENSG00000198914] (nonsyndromic hearing loss).

In some embodiments, the Replacement domain is derived or isolated from the Target RNA.

In some embodiments, the Intronic Domains carry binding sites that are preferentially-targeted by RNA-binding proteins with disease-causing mutations. In some embodiments, the dissociation constant of these mutated RNA-binding proteins and the Intronic Domain is lower than the dissociation constant of the non-mutated RNA-binding protein and the Intronic Domain.

In some embodiments, the trans-splicing RNA further comprises a 5′ untranslated region. In some embodiments, the 5′ untranslated region increases the stability of the trans-splicing nucleic acid. In some embodiments, the 5′ untranslated region reduces the stability of the trans-splicing nucleic acid. In some embodiments, the 5′ untranslated region alters the localization of the trans-splicing nucleic acid. In some embodiments, the 5′ untranslated region alters the processing of the trans-splicing nucleic acid.

In some embodiments, the trans-splicing RNA further comprises a 3′ untranslated region. In some embodiments, the 3′ untranslated region increases the stability of the trans-splicing nucleic acid. In some embodiments, the 3′ untranslated region reduces the stability of the trans-splicing nucleic acid. In some embodiments, the 3′ untranslated region alters the localization of the trans-splicing nucleic acid. In some embodiments, the 3′ untranslated region alters the processing of the trans-splicing nucleic acid.

In some embodiments, the Replacement Domain is comprised of sequence derived or isolated from a human gene. In some embodiments of the compositions of the disclosure, the sequence comprising the Replacement Domain has at least 50%, 55%, 60%, 65%, 70%, 75%, 80%, 87%, 90%, 95%, 97%, 99% or any percentage in between of identity with a human gene. In some embodiments, the Replacement Domain has 100% identity with a sequence derived or isolated from a human gene. In some embodiments, the Replacement Domain comprises or consists of 2 nucleotides, 5 nucleotides, 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between.

In some embodiments of the compositions of the disclosure, the sequence comprising the Antisense Domain has at least 50%, 55%, 60%, 65%, 70%, 75%, 80%, 87%, 90%, 91%, 92%, 93%, 94%, 95%, 96%, 97%, 98%, 99% or any percentage in between of complementarity to the Target RNA sequence. In some embodiments, the Antisense Domain has 100% complementarity to the Target RNA sequence. In some embodiments, the Antisense Domain comprises or consists of 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between the complementary to the Target RNA sequence.

In some embodiments of the compositions of the disclosure, the sequence encoding the trans-splicing RNA further comprises a sequence encoding a promoter capable of expressing the trans-splicing RNA in a eukaryotic cell.

In some embodiments of the compositions of the disclosure, the eukaryotic cell is an animal cell. In some embodiments, the animal cell is a mammalian cell. In some embodiments, the animal cell is a human cell.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex, an exosome or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, 5′ or 3′ untranslated regions, and introns.

Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, H1 and U6 promoters. In some embodiments, the promoter is a sequence isolated or derived from a promoter capable of driving expression of a transfer RNA (tRNA). In some embodiments, the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter is isolated or derived from a valine tRNA promoter.

An “enhancer” is a region of DNA that can be bound by activating proteins to increase the likelihood or frequency of transcription. Non-limiting exemplary enhancers and post-transcriptional regulatory elements include the CMV enhancer and WPRE.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 0.3 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh74 vector, a modified AAV.rh74 vector, an AAV.rh64R1 vector, and a modified AAV.rh64R1 vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2): 132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments, the trans-splicing nucleic acid is RNA, DNA, a DNA/RNA hybrid, and/or comprises at least one of a nucleic acid analog, a chemically-modified nucleic acid, or a chimera composed of two or more nucleic acids or nucleic acid analogs. As used herein, the term “nucleic acid analog” refers to a compound having structural similarity to a canonical purine or pyrimidine base occurring in DNA or RNA. The nucleic acid analog may contain a modified sugar and/or a modified nucleobase, as compared to a purine or pyrimidine base occurring naturally in DNA or RNA. In some embodiments, the nucleic acid analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the nucleic acid analog may be selected from the group consisting of inosine, 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In other embodiments the nucleic acid analog is a nucleic acid mimic (such as, for example, artificial nucleic acids and xeno nucleic acids (XNA).

### BRIEF DESCRIPTION OF THE DRAWINGS

FIGS. 1A-1C illustrate the unmet need addressed by the present invention and provides a schematic of the invention. FIG. 1A illustrates the concept of human genetic disease where mutated (“defective”) DNA sequences are transcribed into RNA which directly contribute to disease (“RNA pathogenicity”) or are translated into disease-causing protein (“translation of pathogenic protein”). FIG. 1B illustrates the state-of-the-art RNA trans-splicing technology where a mutation-carrying RNA molecule is targeted by a trans-splicing RNA that corrects the mutation but with low efficiency. This low efficiency is typically insufficient to halt or reverse progression of disease. FIG. 1C illustrates the present invention where the trans-splicing molecule carries trans-splicing enhancer sequences that increase the efficiency of the trans-splicing reaction. This supports halting or reversal of disease progression and/or elimination of key disease phenotypes thereby providing an effective therapeutic for human genetic disease.

FIGS. 2A-2C illustrate three embodiments of the trans-splicing RNA described in this disclosure. FIG. 2A describes a double trans-splicing molecule which carries two antisense domains, one replacement domain, two intronic domains, and at least two trans-splicing enhancer sequences within the intronic domains. This design promotes replacement of an internal sequence within the target RNA while maintaining the adjacent 5′ and 3′ sequences around the replaced sequence. FIGS. 2B and 2C describe terminal trans-splicing molecules that both contain one antisense domain, one replacement domain, one intronic domain, and at least one trans-splicing enhancer sequence within the intronic domain. FIG. 2B illustrates the design of a 3′ terminal trans-splicing RNA that will replace the 3′ terminal end of a target RNA while maintaining the 5′ end. FIG. 2C illustrates the design of a 5′ terminal trans-splicing molecule that will replace the 5′ terminal end of a target RNA while maintaining the 3′ end.

FIGS. 3A-3D illustrate an experiment designed to reveal the importance of trans-splicing enhancer sequences in the context of internal trans-splicing via production of GFP protein. FIG. 3A illustrates the design of a split GFP reporter that carries N- and C-terminal portions of GFP (“N-GFP” and “C-GFP”) but lacks an internal GFP sequence required for fluorescence. In the reporter, this internal sequence is replaced by a short exon with a stop codon that is flanked by introns. The internal sequence (“int-GFP”) is the replacement sequence within an RNA trans-splicing molecule that is flanked by two intronic sequences and two antisense sequences. FIG. 3B illustrates the activity of the reporter alone so that cis-splicing produces a GFP sequence interrupted by a stop codon therefore producing no GFP signal. FIG. 3C illustrates the activity of the reporter in the presence of the trans-splicing molecule without inclusion of trans-splicing enhancer sequences in the trans-splicing molecule so that similarly cis-splicing occurs primarily and GFP signal is not produced. FIG. 3D illustrates the activity of the reporter in the presence of the trans-splicing molecule with inclusion of trans-splicing enhancer sequences so that trans-splicing occurs primarily and GFP signal is produced.

FIGS. 4A-4D illustrate an experiment designed to reveal the importance of trans-splicing enhancer sequences in the context of 5′ terminal trans-splicing. FIG. 4A illustrates the design of a split GFP reporter that carries a C-terminal portion of GFP (“C-GFP”) but lacks an N-terminal GFP sequence required for fluorescence. In the reporter, this N-terminal GFP sequence is replaced by a short exon with a stop codon that is flanked by introns. The N-terminal sequence (“N-GFP”) is the replacement sequence within an RNA trans-splicing molecule that is flanked by one intronic sequence and one antisense sequence. FIG. 4B illustrates the activity of the reporter alone so that cis-splicing produces a GFP sequence interrupted by a stop codon therefore producing no GFP signal. FIG. 4C illustrates the activity of the reporter in the presence of the trans-splicing molecule without inclusion of trans-splicing enhancer sequences in the trans-splicing molecule so that similarly cis-splicing occurs primarily and GFP signal is not produced. FIG. 4D illustrates the activity of the reporter in the presence of the trans-splicing molecule with inclusion of trans-splicing enhancer sequences so that trans-splicing occurs primarily and GFP signal is produced.

FIGS. 5A-5D illustrate an experiment designed to reveal the importance of trans-splicing enhancer sequences in the context of 3′ terminal trans-splicing. FIG. 5A illustrates the design of a split GFP reporter that carries a N-terminal portion of GFP (“N-GFP”) but lacks an C-terminal GFP sequence required for fluorescence. In the reporter, this C-terminal GFP sequence is replaced by a short exon with a stop codon that is flanked by introns. The C-terminal sequence (“C-GFP”) is the replacement sequence within an RNA trans-splicing molecule that is flanked by one intronic sequence and one antisense sequence. FIG. 5B illustrates the activity of the reporter alone so that cis-splicing produces a GFP sequence interrupted by a stop codon therefore producing no GFP signal. FIG. 5C illustrates the activity of the reporter in the presence of the trans-splicing molecule without inclusion of trans-splicing enhancer sequences in the trans-splicing molecule so that similarly cis-splicing occurs primarily and GFP signal is not produced. FIG. 5D illustrates the activity of the reporter in the presence of the trans-splicing molecule with inclusion of trans-splicing enhancer sequences so that trans-splicing occurs primarily and GFP signal is produced.

FIGS. 6A-6B illustrate the molecular pathology associated with myotonic dystrophy type I and a treatment method for this disease involving a trans-splicing therapeutic. FIG. 6A illustrates that myotonic dystrophy type I (DM1) is caused by a ‘CTG’ repeat expansion in DNA which is transcribed into an RNA composed of repeating ‘CUG’ units. This repetitive RNA preferentially binds a splicing factor protein called MBNL1. The resulting titration of MBNL1 from its typical activities causes widespread dysfunctional RNA splicing in the cell. This dysfunctional splicing is responsible for many pathologies associated with this disease including the characteristic myotonia. FIG. 6B illustrates the activity of a trans-splicing therapeutic that addresses this molecular pathology via amplification of MBNL1 protein production. Specifically, a trans-splicing RNA attaches a gene expression-amplifying sequence to the 3′ terminus of MBNL1 mRNA and increases MBNL1 expression. The resulting increase in MBNL1 levels reconstitutes its splicing activity and reverses disease.

FIGS. 7A-7D illustrate an experiment designed to reveal the importance of trans-splicing enhancer sequences in the context of 3′ terminal trans-splicing for the purpose of increasing gene expression via production of luciferase protein. FIG. 7A illustrates the design of a luciferase reporter that carries a Renilla luciferase control (“R. luciferase”) and a Firefly luciferase (“F. luciferase”) reporter molecule. The reporter molecule carries exon 9, intron 9, and exon 10 from the human MBNL1 gene. Exon 10 contains regulatory elements that influence MBNL1 gene expression. The trans-splicing RNA targets intron 9 and replaces exon 10 with a translation enhancer in a 3′ trans-splicing process. In this manner, successful trans-splicing increases the production of Firefly luciferase relative to Renilla luciferase. FIG. 7B illustrates the activity of the reporter alone so that cis-splicing yields a luciferase molecule with exon 10 of MBNL1. FIG. 7C illustrates the activity of the reporter in the presence of the trans-splicing molecule without inclusion of trans-splicing enhancer sequences in the trans-splicing molecule so that similarly cis-splicing occurs primarily yielding a luciferase molecule with exon 10 of MBNL1. FIG. 7D illustrates the activity of the reporter in the presence of the trans-splicing molecule with inclusion of trans-splicing enhancer sequences so that trans-splicing occurs primarily and exon 10 is replaced by a translation enhancer, therefore increasing Firefly luciferase signal.

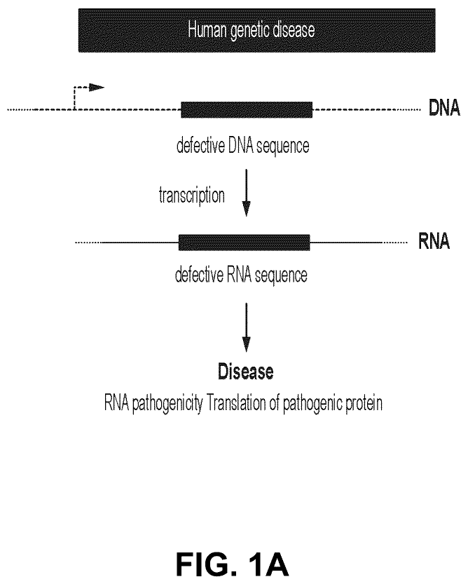

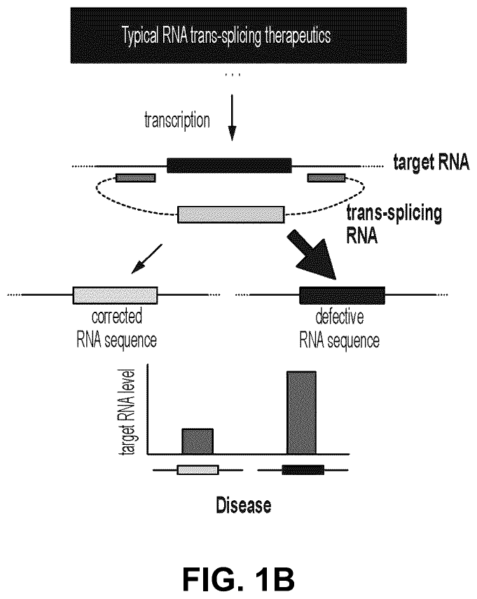

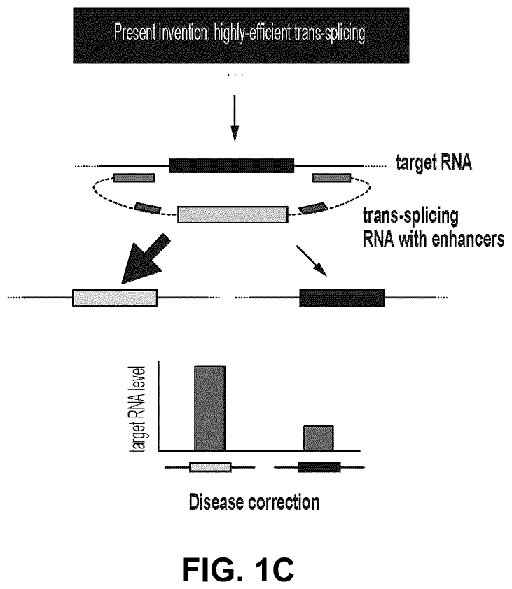

### DETAILED DESCRIPTION

The disclosure provides an RNA molecule that selectively binds and promotes a trans-splicing reaction with an RNA molecule with high efficiency. The disclosure provides vectors, compositions and cells comprising or encoding the trans-splicing RNA molecule. The disclosure provides methods of using the trans-splicing RNA molecule, vectors, compositions and cells of the disclosure to treat a disease or disorder.

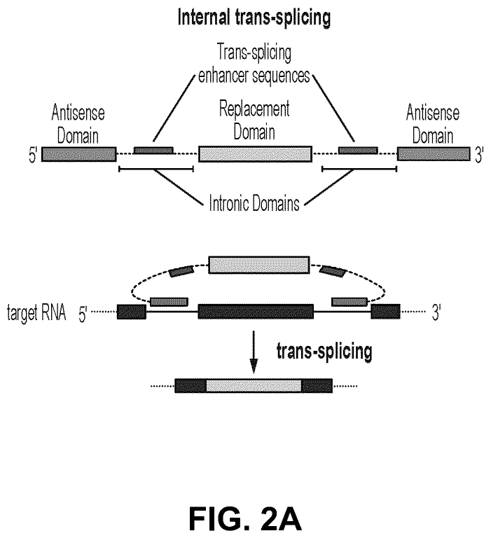

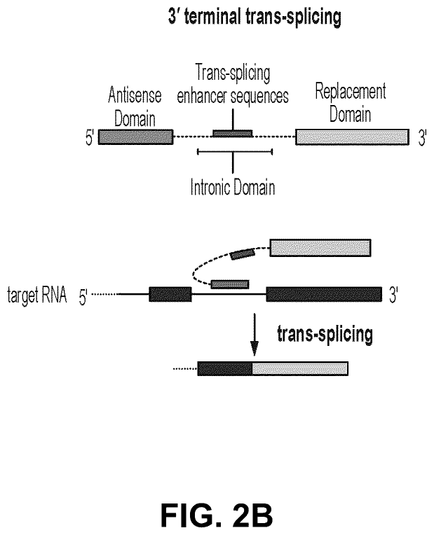

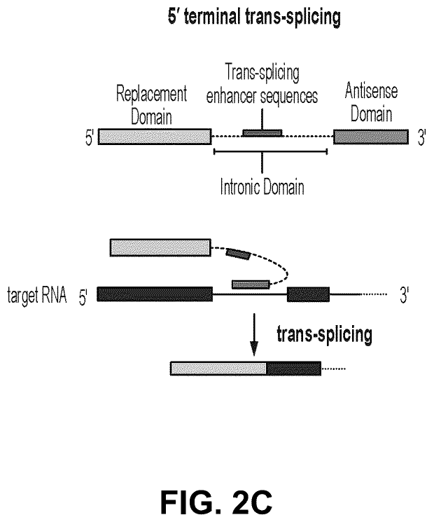

In one aspect, the invention is a trans-splicing RNA molecule comprising three types of domains (FIGS. 2A-2C). One of the three domain types is the Replacement Domain which is inserted into a Target RNA molecule via a trans-splicing reaction. A second domain type is the Antisense Domain which is complementary to a Target RNA. A third domain type is the Intronic Domain which promotes the trans-splicing reaction between the trans-splicing RNA molecule and the Target RNA. The Intronic Domain further comprises intronic trans-splicing enhancing sequences (trans-splicing enhancer sequences) that promote the trans-splicing reaction. This novel combination of specific trans-splicing enhancer sequences and the Intronic Domain promotes RNA trans-splicing in a manner that is sufficiently to replace disease-causing RNA sequences in human cells to address disease. The disclosure provides compositions and methods for specifically targeting disease-causing RNA molecules and replacing disease-causing RNA sequences within these RNA molecules with high efficiency. The trans-splicing RNA molecule implementations show utility in a variety of contexts including replacement of disease-causing sequences or insertion of engineered sequences into Target RNAs. The engineered sequences can alter the translation or stability of Target RNAs to increase or decrease protein production or Target RNA levels. This disclosure provides vectors, compositions and cells comprising or encoding the trans-splicing RNA and methods of using the trans-splicing RNA compositions.

In one aspect, the invention is an RNA technology that enables replacement of arbitrary sequences within specific RNA molecules in living cells. The technology, based on RNA trans-splicing, utilizes the naturally-existing spliceosome in human cells to provide the catalytic activity for this trans-splicing process Typically, RNA splicing occurs within RNA molecules where exons are concatenated and introns removed from immature messenger RNA molecules (pre-mRNAs) to form mature messenger RNA molecules (mRNAs). This process is referred to as cis-splicing. RNA trans-splicing is a process by which the spliceosome concatenates exons derived from distinct and separate RNA molecules. This process rarely occurs in human cells and state-of-the-art systems that promote RNA trans-splicing are active at low levels. The present invention comprises compositions that increase the efficiency of RNA trans-splicing. These improved RNA trans-splicing compositions could be used to replace mutated sequences within a target RNA molecule to address a human disease. Replacement of arbitrary RNA sequences is a general ability with innumerable specific applications a few of which have been explored as relevant demonstrations. RNA trans-splicing can insert engineered sequences into a target RNA to impart new activities to the target RNA such as altered RNA stability or altered RNA translation. This feature can be used to increase production of protein by a target RNA. In the broadest sense, this RNA trans-splicing technology can impart arbitrary changes to both coding and non-coding regions of target RNAs.

References describe the activity of splicing enhancing-sequences in the context of cis-splicing. But little is known of: 1) the identity of trans-splicing enhancing sequences, and 2) whether cis-splicing enhancers also enhance trans-splicing. As the kinetics of intermolecular (trans-splicing) rather than intramolecular (cis-splicing) reactions are likely vastly different, the present inventor conceived of a distinct group of splicing enhancers that would function in the context of trans-splicing. This was confirmed by experiments that indicate that activity of splicing enhancers in cis-splicing is not necessarily predictive of activity in trans-splicing. As used herein, these sequences that promote trans-splicing are termed “trans-splicing enhancer sequences”.

Compositions comprising intronic trans-splicing enhancing sequences (trans-splicing enhancer sequences) disclosed herein include any sequences that promote trans-splicing in an efficient manner. Exemplary trans-splicing enhancer sequences include without limitation: TTACGG, TAACGG, GGGTTT, GTTTTG, GGTTTT, GGTTTG, GGTTGG, GTTAGG, TGGTTG, GGGTAG, GGTAGG, GGTAGT, GTAGTT, GTTGGT, GTGGTT, GGTGGT, TGGTGG, TTGGTG, GTAAGG, TAAGGG, TTAGGG, TAGGGG, TTGGGG, GTTGGG, GTAGGG, TATTGG, TGTTGG, TATGGG, TTTGGG, TGTGGG, TTGTGG, GAGTGT, GAGGTA, GGAGGT, TGGGAG, GGGGTG, GGGGGA, GGGGGT, GGGGTA, GGGAGG, GGGTGG, GGAGGG, GGTGGG, GAGGGG, GTGGGG, GAGTGG, GTATGG, GGTATT, GTATTT, GTATTG, AGTTTA, AGGTTA, GTAACG, AGGTAA, GGTAAG, TGGGGG, AGGGTT, AGGTTG, AGGTAG, ATTTGG, AGTTGG, TCTGGG, AGAGTG, AGAGGG, AGTGTG, AGAGGT, AGGGAG, AGGGTG, AGGGGG, AGGGGT, AGTGGG, AGTATG, AGGTAT, GTATTC, GGTAAC. In some embodiments, in the above exemplary trans-splicing enhancer sequences, none, some, or all, of the thymidine bases may be replaced with uracil so that the exemplary trans-splicing enhancer sequences include without limitation: UUACGG, UAACGG, GGGUUU, GUUUUG, GGUUUU, GGUUUG, GGUUGG, GUUAGG, UGGUUG, GGGUAG, GGUAGG, GGUAGU, GUAGUU, GUUGGU, GUGGUU, GGUGGU, UGGUGG, UUGGUG, GUAAGG, UAAGGG, UUAGGG, UAGGGG, UUGGGG, GUUGGG, GUAGGG, UAUUGG, UGUUGG, UAUGGG, UUUGGG, UGUGGG, UUGUGG, GAGUGU, GAGGUA, GGAGGU, UGGGAG, GGGGUG, GGGGGA, GGGGGU, GGGGUA, GGGAGG, GGGUGG, GGAGGG, GGUGGG, GAGGGG, GUGGGG, GAGUGG, GUAUGG, GGUAUU, GUAUUU, GUAUUG, AGUUUA, AGGUUA, GUAACG, AGGUAA, GGUAAG, UGGGGG, AGGGUU, AGGUUG, AGGUAG, AUUUGG, AGUUGG, UCUGGG, AGAGUG, AGAGGG, AGUGUG, AGAGGU, AGGGAG, AGGGUG, AGGGGG, AGGGGU, AGUGGG, AGUAUG, AGGUAU, GUAUUC, GGUAAC.

The presently disclosed RNA trans-splicing technology which involves the inclusion of specific intronic trans-splicing enhancing sequences (trans-splicing enhancer sequences), is the first to show RNA-trans-splicing with high efficiency against multiple RNA targets. Highly efficient RNA trans-splicing has three primary advantages over previous RNA trans-splicing systems. First, this improved efficiency can replace defective RNA sequences at levels sufficient to reconstitute the activity of mutated genes to treat recessive genetic disorders. Indeed, treatment of many recessive gene disorders require at least 30% efficiency where 100% is complete replacement of a sequence within a Target RNA. Second, this improved efficiency can replace defective RNA sequences at levels sufficient to treat dominant genetic disorders. As a single mutated allele is sufficient to cause disease, many diseases in this class require highly-efficient replacement of mutated sequences as the mutated sequences typically cause toxicity. As a result, even higher efficiency is required (70%+). Finally, the broad ability of our RNA trans-splicing technology to modify multiple Target RNAs demonstrates the first broadly-applicable and efficient version of this technology. This is a very general capability, with this disclosure providing demonstrations of RNA trans-splicing system that can efficiently replace sequences with multiple target RNAs.

The inclusion of intronic trans-splicing enhancing sequences (trans-splicing enhancer sequences) to form the present RNA trans-splicing technology is a general capability that further allows the alteration of non-coding sequences within target RNAs. By replacing the 5′ or 3′ untranslated regions of Target RNAs with high efficiency, this invention allows the alteration of RNA behaviors such as translation or turnover. The net result of these effects is increased production of protein from Target RNAs or other downstream effects associated with altered RNA levels.

### Identification of Intronic Trans-Splicing Enhancing Sequences (Trans-Splicing Enhancer Sequences)

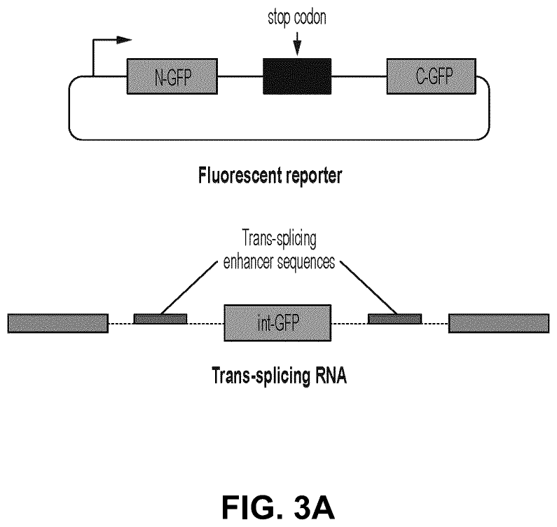

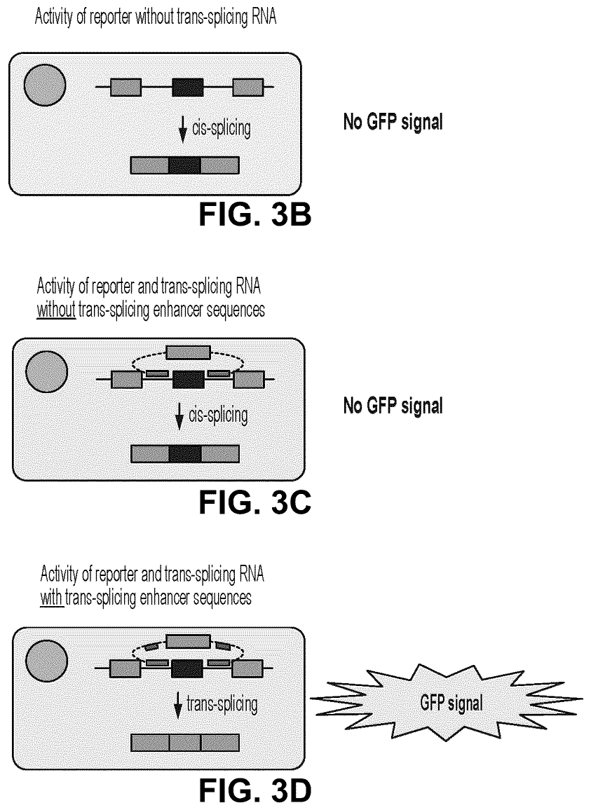

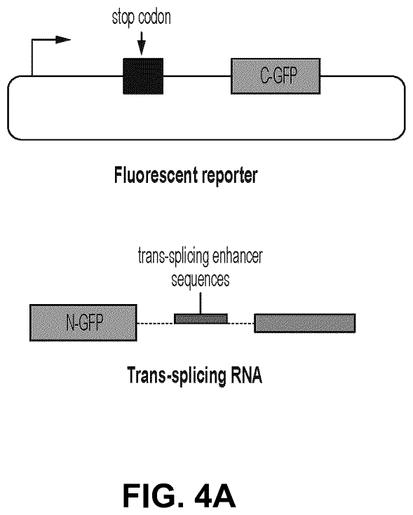

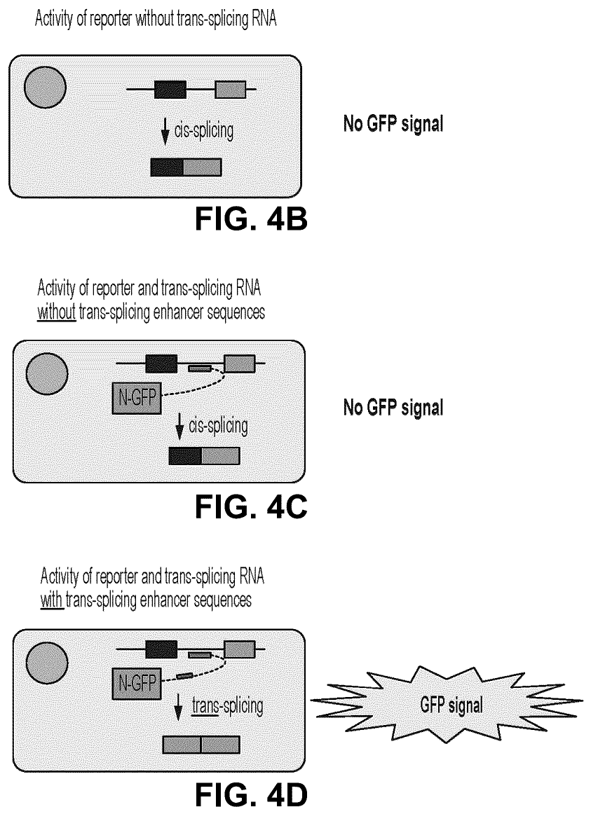

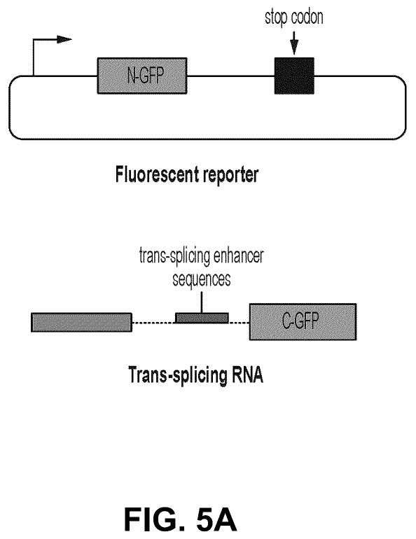

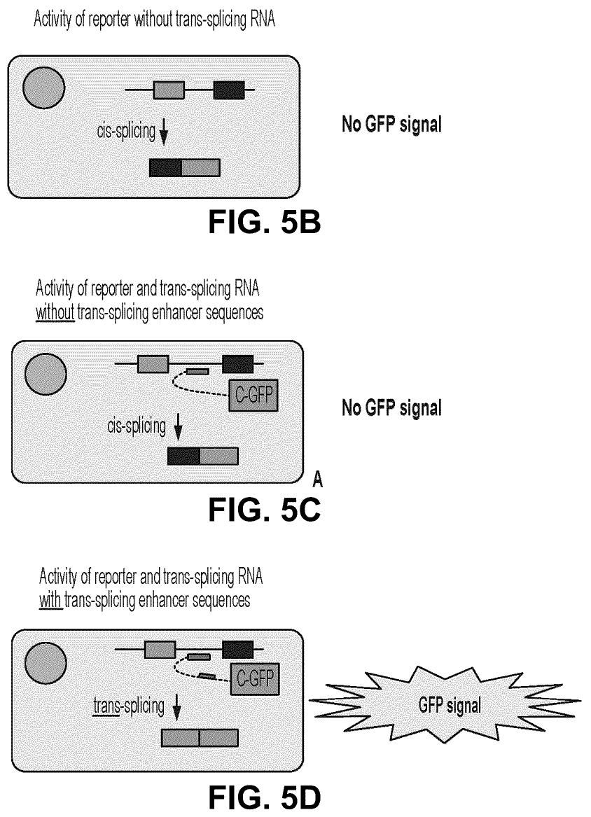

RNA sequences that influence RNA cis-splicing have been known (Wang, Ma et al. 2012). But no study has evaluated the activity of these known splicing enhancers in the context of RNA trans-splicing. The present inventor first systematically assessed whether 50 splicing enhancing sequences could increase the efficiency of RNA-trans-splicing when placed up- and down-stream of the Replacement Sequence in a model RNA trans-splicing molecule. These variant trans-splicing molecules target a split GFP reporter assay that fluoresces only after successful activity of the RNA trans-spicing molecule (FIGS. 3A-3D, 4A-4D, and 5A-5D). This assay is qualitative, not fully quantitative, but is useful because it is what end-users in cell biology often use when attempting to answer scientific questions about the presence, absence, or general magnitude of a transcript. GFP trans-splicing reporters has, accordingly, been widely used in the study of RNA trans-splicing technologies. A GFP reporter similar to a published system (Koller, Wally et al. 2011) was used to compare the relative influence of different trans-splicing enhancer sequences on the efficiency of the trans-splicing reaction.

FIGS. 3A-3D, 4A-4D, and 5A-5D contain a schematic of the plasmids used in the trans-splicing activity assays. Experiments were conducted with either a transiently-transfected reporter and trans-splicing molecule or systems packaged in lentivirus. The present inventor observed that some known splicing enhancing sequences that function in the context of cis-splicing (Wang, Ma et al. 2012) do not enhance trans-splicing. The present inventor further observed that other sequences that may or may not enhance cis-splicing function in the context of trans-splicing. As used herein, these trans-splicing-specific enhancers are termed “trans-splicing enhancer sequences”.

### Use of Intronic Trans-Splicing Enhancing Sequences (Trans-Splicing Enhancer Sequences) to Increase the Translation of Specific Target RNAs

In addition to replacing specific mutated sequences within RNAs with non-mutated sequences, another useful operation on target mRNA molecules is increasing the protein produced by mRNAs. There have been many attempts to address this problem of insufficient protein production from specific mRNAs but each approach has major shortcomings. Indeed, small molecule drugs that increase translation by promoting stop codon read-through suffer extensive off-targets due to promotion of read through on non-target mRNAs (Keeling, Xue et al. 2014). Further, pre-mature stop codons are only one of many causes of insufficient protein levels. Engineered tRNAs to block pre-mature termination codons suffer from this same fundamental issue (WO 2018/161032 A1). An RNA trans-splicing system, in contrast, could replace sequences in any target mRNA with translation-amplifying sequences to increase protein production. The present inventor conceived of efficient RNA trans-splicing mediated by intronic trans-splicing enhancing sequences (trans-splicing enhancer sequences) could address this long-felt but unmet need of a means to promote targeted amplification of protein production from specific mRNAs.

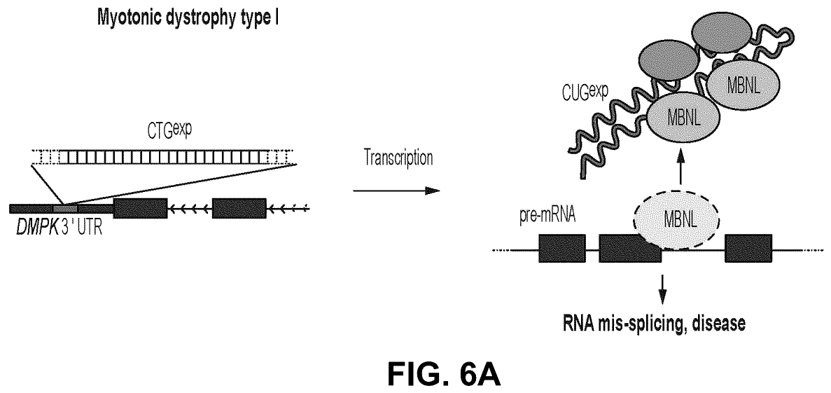

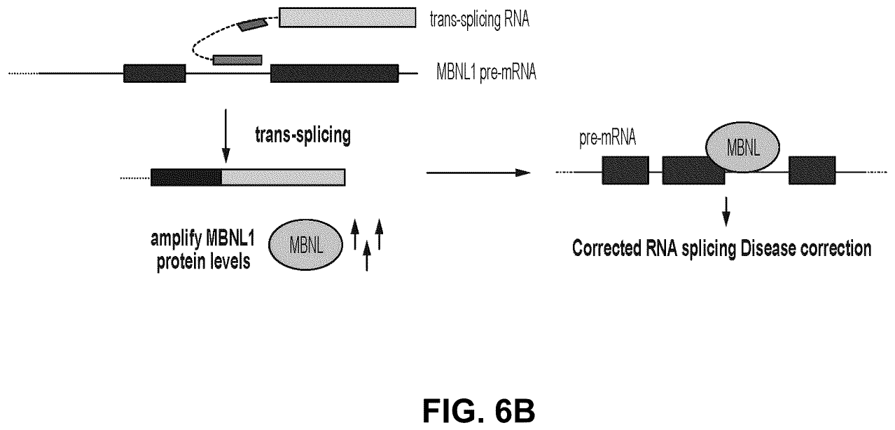

Myotonic dystrophy is caused by RNAs that carry repetitive ‘CUG’ tracts that bind the splicing factor MBNL1. Titration of MBNL1 away from its typical targets causes widespread dysfunction of RNA alternative splicing and is responsible for most manifestations of disease in patients. The present inventor conceived of increasing MBNL1 protein production with an efficient RNA trans-splicing approach could address this disease via production of sufficient MBNL1 protein to reconstitute its typical activities in alternative splicing regulation (FIGS. 6A-6B).

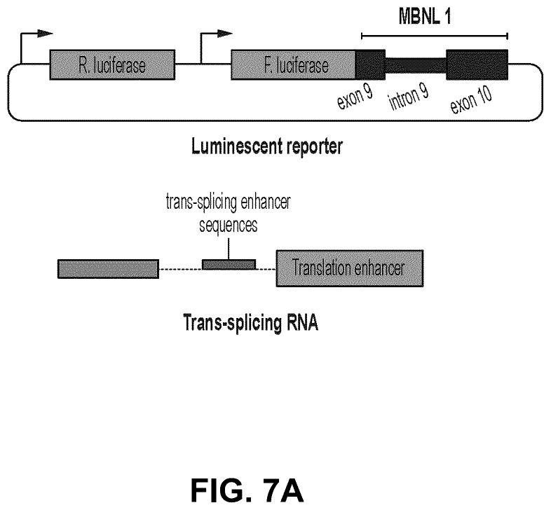

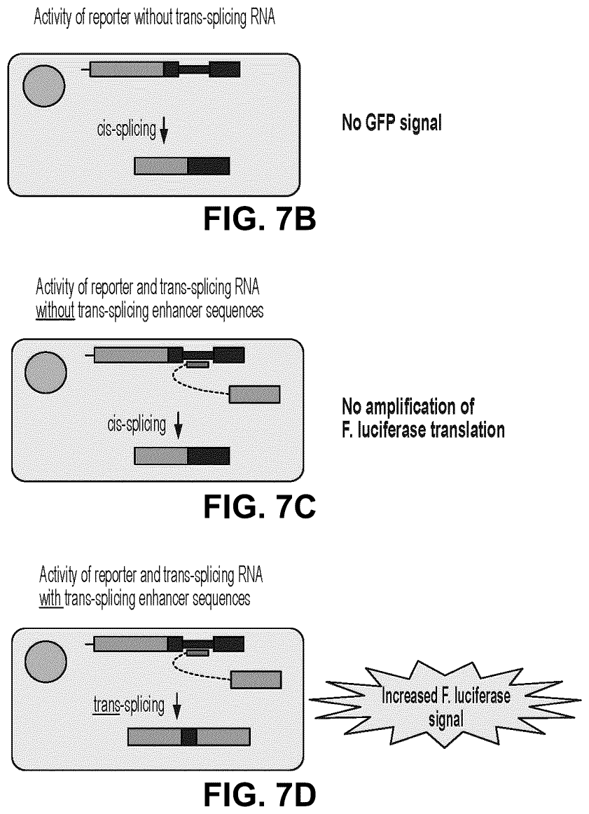

To assess the ability of an RNA trans-splicing systems containing trans-splicing enhancer sequences to increase protein production from specific mRNAs, The present inventor created an RNA trans-splicing system carrying various cis-splicing enhancer sequences and a Woodchuck Hepatitis Virus (WHV) post-transcriptional Regulatory Element (WPRE) (FIGS. 7A-7D). The present inventor also created a reporter that contains a firefly luciferase coding sequence and the last 2 exons and intervening intron of MBNL1 (FIGS. 7A-7D). This assay is qualitative, not fully quantitative, but is useful because it is what end-users in cell biology often use when attempting to answer scientific questions about the presence, absence, or general magnitude of a transcript. Indeed, this reporter is based on the pMIR-GLO luciferase vector that is typically used to assess the stability and protein production from a model mRNA.

Experiments were conducted with either transiently-transfected reporter and trans-splicing molecule or systems packaged in lentivirus. The present inventor observed that some known splicing enhancing sequences that function in the context of cis-splicing (Wang, Ma et al. 2012) do not enhance trans-splicing. The present inventor also observed that other sequences that may or may not enhance cis-splicing function in the context of trans-splicing. As used herein, these trans-splicing-specific enhancers are termed “trans-splicing enhancer sequences”. These results indicate that the use of trans-splicing enhancer sequences can promote significant increases in protein production from a specific mRNA.

### Replacement Domains

Compositions comprising replacement domains disclosed herein include any strategies where replacement or insertion of RNA sequences could be an effective therapy. Exemplary replacement domains include without limitation sequences derived or isolated from the following genes (with gene accession IDs in brackets and associated diseases in parentheses) such as TNFRSF13B [ENSG00000240505] (common variable immune deficiency); ADA, CECR1 [ENSG00000196839, ENSG00000093072] (Adenosine deaminase deficiency); IL2RG [ENSG00000147168] (X-linked severe combined immunodeficiency); HBB [ENSG00000244734] (Beta-thassalemia); HBA1, HBA2 [ENSG00000206172, ENSG00000188536] (alpha-thassalemia); U2AF1 [ENSG00000160201] (myelodysplastic syndrome); SOD1, TARDBP, FUS, MATR3, SOD1, C9ORF72 [ENSG00000142168, ENSG00000120948, ENSG00000089280, ENSG00000015479, ENSG00000142168, ENSG00000147894] (Amyotrophic lateral sclerosis); MAPT, PGRN [ENSG00000186868, ENSG00000030582] (Frontotemporal dementia with parkinsonism); CDH23, MYO7A, USH2A [ENSG00000107736, ENSG00000137474, ENSG00000042781] (Usher's syndrome); GALC [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINK1, ATPGAP2 [ENSG00000158828] (early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466] (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646] (genetic epilepsy disorders); ATM [ENSG00000149311] (Ataxia-telangiectasia); GLB1 [ENSG00000170266] (GM1 gangliosidosis); GBA [ENSG00000177628] (Gaucher disease); GM2A [ENSG00000196743] (GM2 gangliosidosis); UBE3A [ENSG00000114062] (Angelman syndrome); SLC2A1 [ENSG00000117394] (glucose transporter deficiency type 1); LAMP2 [ENSG00000005893] (Danon disease); GLA [ENSG00000102393] (Fabry disease); PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG00000130203] (Familial hypercholesterolemia); MYOC, OPTN, TBK1, WDR36, CYPIB1 [ENSG00000034971, ENSG00000123240, ENSG00000183735, ENSG00000134987, ENSG00000138061] (Open Angle Glaucoma); IDUA [ENSG00000127415] (Hurler syndrome or Mucopolysaccharidosis 1); IDS [ENSG00000010404] (Hunter syndrome or Mucopolysaccharidosis 2); CLN3 [ENSG00000188603] (Batten disease); DMD [ENSG00000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SGCB [ENSG00000163069] (Limb-girdle muscular dystrophy type 2E); SGCG [ENSG00000102683] (Limb-girdle muscular dystrophy type 2C); SGCD [ENSG00000170624] (Limb-girdle muscular dystrophy type 2F); DUX4 [ENSG00000260596] (Facioscapulohumeral muscular dystrophy); F9 [ENSG00000101981] (Hemophilia B); F8 [ENSG00000185010] (Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DFNA1, WFS1 [ENSG00000165474, ENSG00000121742, ENSG00000242866, ENSG00000131504, ENSG00000109501] (autosomal dominant hearing impairment); POU3F3 [ENSG00000198914] (nonsyndromic hearing loss).

In some embodiments, the replacement domain is codon optimized.

In addition to sequences derived from human genes, Replacement Domains can comprise sequences derived from other organisms in order to alter the stability, translation, processing, or localization of a target RNA. Exemplary replacement domains derived from non-human sources include without limitation sequences that increase protein production such as those derived or isolated from Woodchuck Hepatitis Virus (WHV) Post-transcriptional Regulatory Element (WPRE), triplex from MALAT1, the PRE of Hepatitis B virus (HPRE), and an iron response element of the form CAGYCX (Y=U or A; X=U, C, or A).

### Antisense Domains

In some embodiments of the compositions of the disclosure, a pathogenic RNA molecule is a target RNA. In some embodiments, the target RNA comprises a target sequence that is complementary to an antisense domain of the trans-splicing RNA of the disclosure.

In some embodiments of the compositions and methods of the disclosure, the target sequence comprises or consists of between 5 and 500 nucleotides. In some embodiments, the target sequence comprises or consists of between 50 and 250 nucleotides. In some embodiments, the target sequence comprises or consists of between 5 and 50 nucleotides.

In some embodiments of the compositions and methods of the disclosure, a target sequence is contained within a single contiguous stretch of the target RNA. In some embodiments, the target sequence may consist of comprise of one or more nucleotides that are not spread among a single contiguous stretch of the target RNA.

In some embodiments of the disclosure, an Antisense Domain of the disclosure binds to a target sequence. In some embodiments of the disclosure, an antisense domain of the disclosure binds to a target RNA.

In some embodiments of the disclosure, the Antisense Domain is chosen so that successful trans-splicing causes removal of micro open reading frames in the Target RNA. In this manner, the trans-splicing system removes micro open reading frames and increases the production of protein from the target RNA.

### Vectors

In some embodiments of the compositions and methods of the disclosure, a vector comprises or encodes a trans-splicing nucleic acid of the disclosure. In some embodiments, the vector comprises or encodes at least one trans-splicing nucleic acid of the disclosure. In some embodiments, the vector comprises or encodes one or more trans-splicing nucleic acid(s) of the disclosure. In some embodiments, the vector comprises or encodes two or more trans-splicing nucleic acids of the disclosure.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term“recombinant expression system” refers to a genetic construct for the expression of certain genetic material formed by recombination.

In some embodiments, the liposome, lipoplex, or nanoparticle can further comprise a non-cationic lipid, a PEG conjugated lipid, a sterol, or any combination thereof.

In some embodiments, the the liposome, lipoplex, or nanoparticle further comprises a non-cationic lipid, wherein the non-ionic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine and non-cationic lipids described, for example, in WO2017/099823 or US2018/0028664.

In some embodiments, the liposome, lipoplex, or nanoparticle further comprises a conjugated lipid, wherein the conjugated lipid, wherein the conjugated-lipid is selected from the group consisting of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt.

In some embodiments, the liposome, lipoplex, or nanoparticle further comprises cholesterol or a cholesterol derivative.

In some embodiments, the liposome, lipoplex, or nanoparticle further comprises an ionizable lipid, a non-cationic lipid, a conjugated lipid that inhibits aggregation of particles, and a sterol. The amount of the ionizable lipid, the non-cationic lipid, the conjugated lipid that inhibits aggregation of particles, and the sterol can be varied independently. In some embodiments, the lipid nanoparticle comprises an ionizable lipid in an amount from about 20 mol % to about 90 mol % of the total lipid present in the particle, a non-cationic lipid in an amount from about 5 mol % to about 30 mol % of the total lipid present in the particle, a conjugated lipid that inhibits aggregation of particles in an amount from about 0.5 mol % to about 20 mol % of the total lipid present in the particle, and a sterol in an amount from about 20 mol % to about 50 mol % of the total lipid present in the particle.

The ratio of total lipid to DNA vector can be varied as desired. For example, the total lipid to DNA vector (mass or weight) ratio can be from about 10:1 to about 30:1.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. An“enhancer” is a region of DNA that can be bound by activating proteins to increase the likelihood or frequency of transcription. Non-limiting exemplary enhancers and posttranscriptional regulatory elements include the CMV enhancer and WPRE.

### Nucleic Acids

Also provided herein are nucleic acid sequences encoding the trans-splicing nucleic acids disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or“equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical nucleic acid sequence to the reference nucleic acid sequence when compared using sequence identity methods run under default conditions. Specific sequences are provided as examples of particular embodiments. Additionally, an equivalent polynucleotide is one that hybridizes under stringent conditions to the reference polynucleotide or its complement.

The nucleic acid sequences (e.g., polynucleotide sequences) disclosed herein may be codon-optimized which is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for various replacement domains can be generated. In some embodiments, such a sequence is optimized for expression in a host or target cell, such as a host cell used to express the trans-splicing RNA containing a replacement domain in which the disclosed methods are practiced (such as in a mammalian cell, e.g., a human cell). Codon preferences and codon usage tables for a particular species can be used to engineer isolated nucleic acid molecules encoding a replacement domain (such as one encoding a protein having at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type protein) that takes advantage of the codon usage preferences of that particular species. For example, the replacement domains disclosed herein can be designed to have codons that are preferentially used by a particular organism of interest. In one example, a replacement domain nucleic acid sequence is optimized for expression in human cells, such as one having at least 70%, at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating nucleic acid sequence. In some embodiments, an isolated trans-splicing nucleic acid molecule encoding at least one replacement domain (which can be part of a vector) includes at least one replacement domain coding sequence that is codon optimized for expression in a eukaryotic cell, or at least one replacement domain coding sequence codon optimized for expression in a human cell. In one embodiment, such a codon optimized replacement domain coding sequence has at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a replacement domain having at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same replacement domain protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H.5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC;

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Methods of Use

The disclosure provides a method of modifying the sequence of an RNA molecule or a protein encoded by the RNA molecule comprising contacting the composition and the RNA molecule under conditions suitable for binding and trans-splicing of one or more of the trans-splicing RNAs (or a portion thereof) to the RNA molecule.

The disclosure provides a method of modifying an activity of a protein encoded by an RNA molecule comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 15% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 20% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 30% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 40% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 50% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 60% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 70% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 80% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an RNA molecule or a protein encoded by the RNA molecule with 90% or more efficiency comprising contacting the composition and the RNA molecule under conditions suitable for binding and trans-splicing of one or more of the trans-splicing RNAs (or a portion thereof) to the RNA molecule.

The disclosure provides a method of modifying the sequence of an untranslated region of an RNA molecule comprising contacting the composition and the RNA molecule under conditions suitable for binding and trans-splicing of one or more of the trans-splicing RNAs (or a portion thereof) to the RNA molecule.

The disclosure provides a method of increasing the expression of an RNA by insertion of WPRE or sequences with similar activity comprising contacting the composition and the RNA molecule under conditions suitable for binding and trans-splicing of one or more of the trans-splicing RNAs (or a portion thereof) to the RNA molecule.

The disclosure provides a method of modifying the composition of a protein encoded by a target RNA comprising contacting the composition and a cell comprising the target RNA under conditions suitable for trans-splicing among the composition and the target RNA.

The disclosure provides a method of modifying the composition of a target RNA with efficiency exceeding 20% where 100% constitutes complete replacement of a chosen sequence within the target RNA comprising contacting the composition and a cell comprising the target RNA under conditions suitable for trans-splicing among the composition and the target RNA.

The disclosure provides a method of modifying the composition of a protein encoded by a target RNA with efficiency at or about 20% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a target RNA with efficiency at or about 60% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a protein encoded by a target RNA with efficiency at or about 60% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a target RNA with efficiency at or about 70% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a protein encoded by a target RNA with efficiency at or about 70% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a target RNA with efficiency at or about 80% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a protein encoded by a target RNA with efficiency at or about 80% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a target RNA with efficiency at or about 90% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a protein encoded by a target RNA with efficiency at or about 90% where 100% constitutes complete replacement of a chosen sequence within the Target RNA comprising contacting the composition and a cell comprising the target RNA under conditions suitable for trans-splicing among the composition and the target RNA.

The disclosure provides a method of modifying the composition of a target RNA with high efficiency comprising contacting the composition and a cell comprising the target RNA under conditions suitable for trans-splicing among the composition and the target RNA. In some embodiments, the cell is in vivo, in vitro, ex vivo or in situ. In some embodiments, the composition comprises a vector comprising or encoding a trans-splicing RNA molecule of the disclosure. In some embodiments, the vector is an AAV.

The disclosure provides a method of modifying the composition of a protein encoded by a target RNA with high efficiency comprising contacting the composition and a cell comprising the target RNA under conditions suitable for trans-splicing among the composition and the target RNA. In some embodiments, the cell is in vivo, in vitro, ex vivo or in situ. In some embodiments, the composition comprises a vector comprising or encoding a trans-splicing RNA molecule of the disclosure. In some embodiments, the vector is an AAV.

The disclosure provides a method of treating a disease or disorder comprising administering to a subject a therapeutically effective amount of a composition of the disclosure.

The disclosure provides a method of treating a disease or disorder comprising administering to a subject a therapeutically effective amount of a composition of the disclosure, wherein the composition comprises a vector comprising or encoding a trans-splicing RNA molecule of the disclosure, and wherein the composition modifies a level of expression of an RNA molecule of the disclosure or a protein encoded by the RNA molecule.

The disclosure provides a method of treating a disease or disorder comprising administering to a subject a therapeutically effective amount of a composition of the disclosure, wherein the composition comprises a vector comprising or encoding a trans-splicing RNA molecule of the disclosure and wherein the composition modifies an activity of a protein encoded by an RNA molecule.

In some embodiments of the compositions and methods of the disclosure, a disease or disorder of the disclosure includes, but is not limited to, a genetic disease or disorder. In some embodiments, the genetic disease or disorder is a single-gene disease or disorder. In some embodiments, the single-gene disease or disorder is an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the singe-gene disease or disorder is, but not limited to, common variable immune deficiency, Adenosine deaminase deficiency, X-linked severe combined immunodeficiency, Beta-thassalemia, alpha-thassalemia, myelodysplastic syndrome, Amyotrophic lateral sclerosis,

Frontotemporal dementia with parkinsonism, Usher's syndrome, Krabbe disease, Niemann Pick disease, prion disease, Dravet syndrome, early-onset Parkinson's disease, spinocerebellar ataxias, genetic epilepsy disorders, Ataxia-telangiectasia, GM1 gangliosidosis, Gaucher disease, GM2 gangliosidosis, Angelman syndrome, glucose transporter deficiency type 1, Danon disease, Fabry disease, Autosomal dominant polycystic kidney disease, Pompe disease, Familial hypercholesterolemia, Open Angle Glaucoma, Hurler syndrome or Mucopolysaccharidosis 1, Hunter syndrome or Mucopolysaccharidosis 2, Batten disease, Duchenne muscular dystrophy, Limb-girdle muscular dystrophy type 1B, Limb-girdle muscular dystrophy type 2B, Limb-girdle muscular dystrophy type 2D, Limb-girdle muscular dystrophy type 2E, Limb-girdle muscular dystrophy type 2C, Limb-girdle muscular dystrophy type 2F, Facioscapulohumeral muscular dystrophy, Hemophilia B, Hemophilia A, Retinitis pigmentosa, cystic fibrosis, autosomal dominant hearing impairment, and non-syndromic hearing loss. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to, Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Niemann-Pick disease, spinal muscular atrophy, and Roberts syndrome. In some embodiments, the single-gene disease or disorder is X-linked disease or disorder including, but not limited to, muscular dystrophy, Duchenne muscular dystrophy, Hemophilia, Adrenoleukodystrophy (ALD), Rett syndrome, and Hemophilia A. In some embodiments, the single-gene disease or disorder is a mitochondrial disorder including, but not limited to, Leber's hereditary optic neuropathy.

In some embodiments of the compositions and methods of the disclosure, a disease or disorder of the disclosure includes, but is not limited to, an immune disease or disorder. In some embodiments, the immune disease or disorder is an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis.

In some embodiments of the compositions and methods of the disclosure, a disease or disorder of the disclosure includes, but is not limited to, an inflammatory disease or disorder.

In some embodiments of the compositions and methods of the disclosure, a disease or disorder of the disclosure includes, but is not limited to, a metabolic disease or disorder.

In some embodiments of the compositions and methods of the disclosure, a disease or disorder of the disclosure includes, but is not limited to, a degenerative or a progressive disease or disorder. In some embodiments, the degenerative or a progressive disease or disorder includes, but is not limited to, amyotrophic lateral sclerosis (ALS), Huntington's disease, Alzheimer's disease, and aging.

In some embodiments of the compositions and methods of the disclosure, a disease or disorder of the disclosure includes, but is not limited to, an infectious disease or disorder.

In some embodiments of the compositions and methods of the disclosure, a disease or disorder of the disclosure includes, but is not limited to, a pediatric or a developmental disease or disorder.

In some embodiments of the compositions and methods of the disclosure, a disease or disorder of the disclosure includes, but is not limited to, a cardiovascular disease or disorder.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer includes, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (Head and Neck Cancer), Pheochromocytoma, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sézary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer involves the present of a gene fusion that produces a chimeric RNA with sequences derived from two genes due to a deletion or translocation of DNA. Gene fusions pairs include but are not limited to: MAN2A1 and FER, DNAJB1 and PRKACA, BCR-ABL1, TMPRSS2 and ERG, EWSR1 and FLI1, PML and RARA, EML4 and ALK, KIAA1549 and BRAF, CCDC6 and RET, SS18 and SSX1, RUNX1 and RUNXIT1, PAX3 and FOX01, NCOA4 and RET, ETV6 and RUNX1, FUS and DDIT3, SS18 and SSX2, NPM1 and ALK, KMT2A and AFF1, TCF3 and PBX1, STIL and TAL1, COL1A1 and PDGFB, CRTC1 and MAML2, NAB2 and STAT6, EWSR1 and ATF1, ETV6 and NTRK3, EWSR1 and ERG, EWSR1 and WT1, DNAJB1 and PRKACA, PAX7 and FOXO1, FUS and CREB3L2, CBFAT3 and GLIS2, PAX8 and PPARG, KMT2A and MLLT1, EWSR1 and NR4A3, KMT2A and MLLT3, ASPSCR1 and TFE3, HMGA2 and LPP, JAZF1 and SUZ12, KIF5B and RET, FUS and ERG, SLC45A3 and ERG, NUP214 and ABL1, SET and NUP214, CD74 and ROS1, ETV6 and ABL1, TPM3 and NTRK1, PRKAR1A and RET, EWSR1 and CREB1, KMT2A and AFDN, EWSR1 and DDIT3, CLTC and ALK, ETV6 and PDGFRB, TPM3 and ALK, KMT2A and MLLT10, TMPRSS2 and ETV1, BRD4 and NUTM1, NUP98 and KDM5A, RANBP2 and ALK, CTNNB1 and PLAG1, KMT2A and ELL, TAF15 and NR4A3, FGFR3 and TACC3, PCM1 and JAK2, YWHAE and NUTM2B, STRN and ALK, CRTC3 and MAML2, CDH11 and USP6, CDKN2D and WDFY2, CIC and DUX4, SLC34A2 and ROS1, ATIC and ALK, CD74 and NRG1, MYB and NFIB, PRCC and TFE3, KIF5B and ALK, TMPRSS2 and ETV4, KMT2A and SEPT9, EWSR1 and POU5F1, FGFR1 and PLAG1, MN1 and ETV6, TBL1XR1 and TP63, KMT2A and EPS15, SLC45A3 and ELK4, DHH and RHEBL1, HEY1 and NCOA2, EZR and ROS1, GOPC and ROS1, HMGA2 and WIF1, KMT2A and CREBBP, SS18 and SSX4B, FAM131B and BRAF, EWSR1 and FEV, EWSR1 and PBX1, TPM4 and ALK, SND1 and BRAF, ACTB and GLI1, KMT2A and KNL1, KMT2A and SEPT6, SDC4 and ROS1, TFG and ALK, HNRNPA2B1 and ETV1, PTPRK and RSPO3, JAZF1 and PHF1, HMGA2 and RAD51B, KMT2A and MLLT11, TPR and NTRK1, AKAP9 and BRAF, FUS and CREB3L1, ETV6 and JAK2, HMGA2 and NFIB, KMT2A and AFF3, CHCHD7 and PLAG1, VTI1A and TCF7L2, LIFR and PLAG1, EWSR1 and ETV1, SRGAP3 and RAF1, KMT2A and AFF4, MEAF6 and PHF1, PAX3 and NCOA1, HAS2 and PLAG1, EWSR1 and NFATC2, HIP1 and ALK, GOLGA5 and RET, BCR and JAK2, EWSR1 and ETV4, DCTN1 and ALK, MBTD1 and CXorf67, NDRG1 and ERG, CARS and ALK, SFPQ and TFE3, KMT2A and ARHGAP26, KMT2A and EP300, KMT2A and TET1, PAX5 and JAK2, PPFIBP1 and ALK, YWHAE and NUTM2A, LRIG3 and ROS1, TFG and NTRK1, TPM3 and ROS1, SLC45A3 and ETV1, ERC1 and RET, SEC16A and NOTCH1, KTN1 and RET, SEC31A and JAK2, TCEA1 and PLAG1, QKI and NTRK2, RNF130 and BRAF, EIF3E and RSPO2, EWSR1 and ZNF444, LMNA and NTRK1, PPFIBP1 and ROS1, PWWP2A and ROS1, EWSR1 and YY1, FUS and ATF1, PAX3 and NCOA2, ZC3H7B and BCOR, BRD3 and NUTM1, CANT1 and ETV4, CIC and FOXO4, COL1A1 and USP6, EWSR1 and ZNF384, KMT2A and ABI1, KMT2A and ACTN4, KMT2A and CEP170B, KMT2A and FOXO3, KMT2A and GAS7, KMT2A and MLLT6, KMT2A and SEPT2, KMT2A and SEPT5, MSN and ALK, VCL and ALK, EZR and ERBB4, RELCH and RET, SLC3A2 and NRG1, TRIM24 and BRAF, KLC1 and ALK, ARID1A and MAST2, GPBP1L1 and MAST2, NFIX and MAST1, NOTCH1 and GABBR2, TADA2A and MAST1, ZNF700 and MAST1, TRIM24 and RET, TRIM33 and RET, SSBP2 and JAK2, KMT2A and EEFSEC, CLCN6 and BRAF, GNAI1 and BRAF, MKRN1 and BRAF, NACC2 and NTRK2, FGFR1 and TACC1, TRIM27 and RET, HMGA2 and FHIT, HOOK3 and RET, PCM1 and RET, CEP89 and BRAF, CLIP1 and ROS1, ERC1 and ROS1, HLA and A and ROS1, LSM14A and BRAF, MYO5A and ROS1, SHTN1 and ROS1, TP53 and NTRK1, TPM3 and ROS1, ZCCHC8 and ROS1, FGFR3 and BAIAP2L1, KLK2 and ETV1, ACSL3 and ETV1, NUP107 and LGR5, HMGA2 and CCNB1IP1, HMGA2 and COX6C, GATM and BRAF, HACL1 and RAF1, HERPUD1 and BRAF, ZSCAN30 and BRAF, SLC45A3 and BRAF, HMGA2 and LHFPL6, COL1A2 and PLAG1, ESRP1 and RAF1, IRF2BP2 and CDX1, TFG and NR4A3, CLTC and TFE3, EWSR1 and MYB, NONO and TFE3, FCHSD1 and BRAF, HMGA2 and EBF1, ACBD6 and RRP15, AGPAT5 and MCPH1, AGTRAP and BRAF, ARFIP1 and FHDC1, ATG4C and FBXO38, BBS9 and PKD1L1, CENPK and KMT2A, CNBP and USP6, DDX5 and ETV4, EIF3K and CYP39A1, EPC1 and PHF1, ERO1A and FERMT2, ETV6 and ITPR2, EWSR1 and NFATC1, EWSR1 and PATZ1, EWSR1 and SMARCA5, EWSR1 and SP3, FBXL18 and RNF216, FGFR1 and ZNF703, FN1 and ALK, FUS and FEV, GMDS and PDE8B, HMGA2 and ALDH2, IL6R and ATP8B2, INTS4 and GAB2, JPT1 and USH1G, KLK2 and ETV4, KMT2A and ABI2, KMT2A and ARHGEF12, KMT2A and BTBD18, KMT2A and CASP8AP2, KMT2A and CBL, KMT2A and CIP2A, KMT2A and CT45A2, KMT2A and DAB2IP, KMT2A and FOX04, KMT2A and FRYL, KMT2A and GMPS, KMT2A and GPHN, KMT2A and LASP1, KMT2A and LPP, KMT2A and MAPRE1, KMT2A and MYO1F, KMT2A and NCKIPSD, KMT2A and NRIP3, KMT2A and PDS5A, KMT2A and PICALM, KMT2A and PRRC1, KMT2A and SARNP, KMT2A and SH3GL1, KMT2A and SORBS2, KMT2A and TOP3A, KMT2A and ZFYVE19, MBOAT2 and PRKCE, MIA2 and GEMIN2, NF1 and ASIC2, NFIA and EHF, NTN1 and ACLY, OMD and USP6, PLA2R1 and RBMS1, PLXND1 and TMCC1, RAF1 and DAZL, RBM14 and PACS1, RGS22 and SYCP1, SEC31A and ALK, SEPT8 and AFF4, SLC22A1 and CUTA, SLC26A6 and PRKAR2A, SLC45A3 and ETV5, SQSTM1 and ALK, SS18L1 and SSX1, SSH2 and SUZ12, SUSD1 and PTBP3, TCF12 and NR4A3, TECTA and TBCEL, THRAP3 and USP6, TMPRSS2 and ETV5, TPR and ALK, UBE2L3 and KRAS, WDCP and ALK, SS18 and USP6

In some embodiments of the methods of the disclosure, a subject of the disclosure has been diagnosed with the disease or disorder. In some embodiments, the subject of the disclosure presents at least one sign or symptom of the disease or disorder. In some embodiments, the subject has a biomarker predictive of a risk of developing the disease or disorder. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject systemically. In some embodiments, the composition of the disclosure is administered to the subject by an intravenous route. In some embodiments, the composition of the disclosure is administered to the subject by an injection or an infusion.

In some embodiments of the methods of the disclosure, a composition of the disclosure is administered to the subject locally. In some embodiments, the composition of the disclosure is administered to the subject by an intraosseous, intraocular, intracerebrospinal or intraspinal route. In some embodiments, the composition of the disclosure is administered directly to the cerebral spinal fluid of the central nervous system. In some embodiments, the composition of the disclosure is administered directly to a tissue or fluid of the eye and does not have bioavailability outside of ocular structures. In some embodiments, the composition of the disclosure is administered to the subject by an injection or an infusion.

In some embodiments, the compositions comprising the trans-splicing RNAs disclosed herein are formulated as pharmaceutical compositions. Briefly, pharmaceutical compositions for use as disclosed herein may comprise a fusion protein(s) or a polynucleotide encoding the fusion protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for oral, intravenous, topical, enteral, intraocular, and/or parenteral administration. In certain embodiments, the compositions of the present disclosure are formulated for intravenous administration.

### EXAMPLE EMBODIMENTS

Embodiment 1: A composition comprising a trans-splicing nucleic acid, comprising: (a) one or more replacement domains that encode a therapeutic sequence operably linked to; (b) one or more intronic domains that promote RNA splicing of the replacement domain comprising intronic trans-splicing enhancing sequence(s); and (c) one or more antisense domains that promote binding to a target RNA molecule.

Embodiment 2: The composition of embodiment 1, wherein the intronic trans-splicing enhancing sequences (trans-splicing enhancer sequences) are derived or isolated from the group of sequences consisting of: TTACGG, TAACGG, GGGTTT, GTTTTG, GGTTTT, GGTTTG, GGTTGG, GTTAGG, TGGTTG, GGGTAG, GGTAGG, GGTAGT, GTAGTT, GTTGGT, GTGGTT, GGTGGT, TGGTGG, TTGGTG, GTAAGG, TAAGGG, TTAGGG, TAGGGG, TTGGGG, GTTGGG, GTAGGG, TATTGG, TGTTGG, TATGGG, TTTGGG, TGTGGG, TTGTGG, GAGTGT, GAGGTA, GGAGGT, TGGGAG, GGGGTG, GGGGGA, GGGGGT, GGGGTA, GGGAGG, GGGTGG, GGAGGG, GGTGGG, GAGGGG, GTGGGG, GAGTGG, GTATGG, GGTATT, GTATTT, GTATTG, AGTTTA, AGGTTA, GTAACG, AGGTAA, GGTAAG, TGGGGG, AGGGTT, AGGTTG, AGGTAG, ATTTGG, AGTTGG, TCTGGG, AGAGTG, AGAGGG, AGTGTG, AGAGGT, AGGGAG, AGGGTG, AGGGGG, AGGGGT, AGTGGG, AGTATG, AGGTAT, GTATTC, GGTAAC. In some embodiments, in the above exemplary trans-splicing enhancer sequences, none, some, or all, of the thymidine bases may be replaced with uracil so that the exemplary trans-splicing enhancer sequences include without limitation: UUACGG, UAACGG, GGGUUU, GUUUUG, GGUUUU, GGUUUG, GGUUGG, GUUAGG, UGGUUG, GGGUAG, GGUAGG, GGUAGU, GUAGUU, GUUGGU, GUGGUU, GGUGGU, UGGUGG, UUGGUG, GUAAGG, UAAGGG, UUAGGG, UAGGGG, UUGGGG, GUUGGG, GUAGGG, UAUUGG, UGUUGG, UAUGGG, UUUGGG, UGUGGG, UUGUGG, GAGUGU, GAGGUA, GGAGGU, UGGGAG, GGGGUG, GGGGGA, GGGGGU, GGGGUA, GGGAGG, GGGUGG, GGAGGG, GGUGGG, GAGGGG, GUGGGG, GAGUGG, GUAUGG, GGUAUU, GUAUUU, GUAUUG, AGUUUA, AGGUUA, GUAACG, AGGUAA, GGUAAG, UGGGGG, AGGGUU, AGGUUG, AGGUAG, AUUUGG, AGUUGG, UCUGGG, AGAGUG, AGAGGG, AGUGUG, AGAGGU, AGGGAG, AGGGUG, AGGGGG, AGGGGU, AGUGGG, AGUAUG, AGGUAU, GUAUUC, GGUAAC.

Embodiment 3: The composition of embodiment 1, wherein the intronic trans-splicing enhancing sequences (trans-splicing enhancer sequences) consist of a chain of RNA nucleobases comprising at least one RNA motif having the formula X₁X₂X₃X₄X₅X₆ wherein; X₁ is selected from the group including adenine (A), uracil (U) and guanine (G); X₂ is selected from the group including adenine (A), uracil (U) and guanine (G); X₃ is selected from the group including adenine (A), uracil (U) and guanine (G); X₄ is selected from the group including adenine (A), uracil (U), cytosine (C) and guanine (G); X₅ is selected from the group including adenine (A), cytosine (C), uracil (U) and guanine (G); and X₆ is selected from the group including adenine (A), uracil (U) and guanine (G).

Embodiment 4: The composition of embodiment 1, wherein the trans-splicing enhancer sequence consists of a chain of RNA nucleobases comprising at least one RNA motif having the formula X₁X₂X₃X₄X₅X₆ wherein; X₁ is selected from the group including adenine (A), uracil (U) and guanine (G); X₂ is selected from the group including adenine (A), uracil (U) and guanine (G); X₃ is selected from the group including adenine (A), uracil (U) and guanine (G); X₄ is selected from the group including adenine (A), uracil (U) and guanine (G); X₅ is selected from the group including adenine (A), uracil (U) and guanine (G); and X₆ is selected from the group including uracil (U) and guanine (G).

Embodiment 5: The composition of embodiment 1, wherein the trans-splicing enhancer sequence consists of a chain of RNA nucleobases comprising at least one RNA motif having the formula X₁X₂X₃X₄X₅X₆ wherein; X₁ is selected from the group including adenine (A), uracil (U) and guanine (G); X₂ is selected from the group including uracil (U) and guanine (G); X₃ is selected from the group including adenine (A), uracil (U) and guanine (G); X₄ is selected from the group including uracil (U) and guanine (G); X₅ is selected from the group including uracil (U) and guanine (G); and X₆ is selected from the group including uracil (U) and guanine (G).

Embodiment 6: The composition of embodiments 1-5, wherein the replacement domain is derived or isolated from a human gene selected from the group consisting of: GLB1 (GM1 gangliosidosis); GBA (Gaucher disease); GM2A (GM2 gangliosidosis); PCSK9, LDLR, APOB, APOE (Familial hypercholesterolemia); GAA (Pompe disease); MYOC, OPTN, TBK1, WDR36, CYPIB1 (Open Angle Glaucoma); IDS (Hunter syndrome or Mucopolysaccharidosis 2); IDUA (Hurler syndrome or Mucopolysaccharidosis 1); CLN3 (Batten disease); F9 (Hemophilia B); F8 (Hemophilia A), LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SGCD (Limb-girdle muscular dystrophy type 2F); DUX4, D4Z4 (Facioscapulohumeral muscular dystrophy); USHA2A, RPGR, RP2, RHO, PRPF31, USH1F, PRPF3, PRPF6 (Retinitis pigmentosa).

Embodiment 7: The composition of embodiments 1-5, wherein the replacement domain is derived or isolated from an expression-enhancing sequence selected from the group consisting of: Woodchuck Hepatitis Virus (WHV) Post-transcriptional Regulatory Element (WPRE), triplex from MALAT1, the PRE of Hepatitis B virus (HPRE), and an iron response element.

Embodiment 8: The composition of any one of embodiments 1-5, wherein the antisense domain is complementary to sequences derived or isolated from a human gene selected from the group consisting of: TNFRSF13B (common variable immune deficiency), ADA, CECR1 (Adenosine deaminase deficiency), IL2RG (X-linked severe combined immunodeficiency), HBB (Beta-thassalemia), HBA1, HBA2 (alpha-thassalemia), U2AF1 (myelodysplastic syndrome), SOD1, TARDBP, FUS, MATR3, SOD1, C9ORF72 (Amyotrophic lateral sclerosis), MAPT, PGRN (Frontotemporal dementia with parkinsonism), CDH23, MYO7A, USH2A (Usher's syndrome), GALC (Krabbe disease), SMPD1, NPC1, NPC2 (Niemann Pick disease), PRNP (prion disease), SCN1A (Dravet syndrome), PINK1, ATPGAP2 (early-onset Parkinson's disease), ATXN1, ATXN2, ATXN3, PLEKHG4, SPTBN2, CACNA1A, ATXN7, TTBK2, PPP2R2B, KCNC3, PRKCG, ITRP1, TBP, KCND1, FGF14 (spinocerebellar ataxias), SCN1A, SCN2A, CACNA1A, GRIN2B, GRIN2A, MECP2, FOXG1, SLC6A1, PRRT2, PTEN, KCNQ2, KCNQ3, STARD7, CLRN1 (genetic epilepsy disorders), ATM (Ataxia-telangiectasia), GLB1 (GM1 gangliosidosis), GBA (Gaucher disease), GM2A (GM2 gangliosidosis), UBE3A (Angelman syndrome), SLC2A1 (glucose transporter deficiency type 1), LAMP2 (Danon disease), GLA (Fabry disease), PKD1, PKD2 (Autosomal dominant polycystic kidney disease), GAA (Pompe disease), PCSK9, LDLR, APOB, APOE (Familial hypercholesterolemia), MYOC, OPTN, TBK1, WDR36, CYPIB1 (Open Angle Glaucoma), IDUA (Hurler syndrome or Mucopolysaccharidosis 1), IDS (Hunter syndrome or Mucopolysaccharidosis 2), CLN3 (Batten disease), DMD (Duchenne muscular dystrophy), LMNA (Limb-girdle muscular dystrophy type 1B), DYSF (Limb-girdle muscular dystrophy type 2B), SGCA (Limb-girdle muscular dystrophy type 2D), SGCB (Limb-girdle muscular dystrophy type 2E), SGCG (Limb-girdle muscular dystrophy type 2C), SGCD (Limb-girdle muscular dystrophy type 2F), DUX4, D4Z4 (Facioscapulohumeral muscular dystrophy), F9 (Hemophilia B), F8 (Hemophilia A), USHA2A, RPGR, RP2, RHO, PRPF31, USH1F, PRPF3, PRPF6 (Retinitis pigmentosa), CFTR (cystic fibrosis), GJB2, GJB6, STRC, DFNA1, DFNA14 (autosomal dominant hearing impairment), POU3F3 (nonsyndromic hearing loss)

Embodiment 9: The composition of any one of embodiments 1-5, wherein the trans-splicing RNA comprises an untranslated region that alters the localization, processing, or transport of the trans-splicing nucleic acid.

Embodiment 10: the composition of any one of embodiments 1-13, wherein the sequence comprising the trans-splicing nucleic acid comprises a sequence that is bound by an RNA-binding protein that increases the trans-splicing efficiency.

Embodiment 11: the composition of any of one embodiments 1-13, wherein the trans-splicing nucleic acid is RNA, DNA, a DNA/RNA hybrid, nucleic acid analog, a chemically-modified nucleic acid, or a chimera composed of two or more nucleic acids or nucleic acid analogs.

Embodiment 12: the composition of any of one embodiments 1-1, wherein the wherein the trans-splicing nucleic acid molecule further comprises a heterologous promoter.

Embodiment 13: the composition of any of one embodiments 1-13, wherein the promoter is isolated or derived from a promoter capable of driving expression of a transfer RNA (tRNA).

### Cells and Tissues

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell. In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, an immune cell of the disclosure is a lymphocyte. In some embodiments, an immune cell of the disclosure is a T lymphocyte (also referred to herein as a T-cell). Exemplary T-cells of the disclosure include, but are not limited to, naïve T cells, effector T cells, helper T cells, memory T cells, regulatory T cells (Tregs) and Gamma delta T cells. In some embodiments, an immune cell of the disclosure is a B lymphocyte. In some embodiments, an immune cell of the disclosure is a natural killer cell. In some embodiments, an immune cell of the disclosure is an antigen-presenting cell.

In some embodiments of the compositions and methods of the disclosure, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell.

In some embodiments of the compositions and methods of the disclosure, a somatic cell of the disclosure is an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a brain cell of the disclosure is a neuronal cell. In some embodiments, a neuron cell of the disclosure is a neuron of the central nervous system. In some embodiments, a neuron cell of the disclosure is a neuron of the brain or the spinal cord. In some embodiments, a neuron cell of the disclosure is a neuron of a cranial nerve or an optic nerve. In some embodiments, a neuron cell of the disclosure is a neuron of the peripheral nervous system. In some embodiments, a neuron cell of the disclosure is a neuroglial or a glial cell. In some embodiments, a glial of the disclosure is a glial cell of the central nervous system including, but not limited to, oligodendrocytes, astrocytes, ependymal cells, and microglia. In some embodiments, a glial of the disclosure is a glial cell of the peripheral nervous system including, but not limited to, Schwann cells and satellite cells.

In some embodiments of the compositions and methods of the disclosure, a liver cell of the disclosure is a hepatocytes. In some embodiments, a liver cell of the disclosure is a hepatic stellate cell. In some embodiments, a liver cell of the disclosure is Kupffer cell. In some embodiments, a liver cell of the disclosure is a sinusoidal endothelial cells.

In some embodiments of the compositions and methods of the disclosure, a retinal cell of the disclosure is a photoreceptor. In some embodiments, a photoreceptor cell of the disclosure is a rod. In some embodiments, a retinal cell of the disclosure is cone. In some embodiments, a retinal cell of the disclosure is a bipolar cell. In some embodiments, a retinal cell of the disclosure is a ganglion cell. In some embodiments, a retinal cell of the disclosure is a horizontal cell. In some embodiments, a retinal cell of the disclosure is an amacrine cell.

In some embodiments of the compositions and methods of the disclosure, a heart cell of the disclosure is a cardiomyocyte. In some embodiments, a heart cell of the disclosure is a cardiac pacemaker cell.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodimented herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

### BIBLIOGRAPHY

Keeling, K. M., X. Xue, G. Gunn and D. M. Bedwell (2014). “Therapeutics based on stop codon readthrough.” Annu Rev Genomics Hum Genet 15: 371-394.

Koller, U., V. Wally, L. G. Mitchell, A. Klausegger, E. M. Murauer, E. Mayr, C. Gruber, S. Hainzl, H. Hintner and J. W. Bauer (2011). “A novel screening system improves genetic correction by internal exon replacement.” Nucleic Acids Res 39(16): e108.

Wang, Y., M. Ma, X. Xiao and Z. Wang (2012). “Intronic splicing enhancers, cognate splicing factors and context-dependent regulation rules.” Nat Struct Mol Biol 19(10): 1044-1052.

1. A composition comprising a trans-splicing nucleic acid, comprising:
(a) one or more replacement domains that encode a therapeutic sequence operably linked to;
(b) one or more intronic domains that promote RNA splicing of the one or more replacement domains, wherein the one or more intronic domains each comprises a trans-splicing enhancer sequence; and
(c) one or more antisense domains that promote binding to a target RNA molecule.

2. The composition of claim 1, wherein the trans-splicing enhancer sequence consists of a chain of RNA nucleobases comprising at least one RNA motif having the formula X₁X₂X₃X₄X₅X₆, wherein:
X₁ is selected from the group consisting of adenine (A), uracil (U) and guanine (G);
X₂ is selected from the group consisting of adenine (A), uracil (U) and guanine (G);
X₃ is selected from the group consisting of adenine (A), uracil (U) and guanine (G);
X₄ is selected from the group consisting of adenine (A), uracil (U), cytosine (C) and guanine (G);
X₅ is selected from the group consisting of adenine (A), cytosine (C), uracil (U) and guanine (G); and
X₆ is selected from the group consisting of adenine (A), uracil (U) and guanine (G).

3. The composition of claim 1, wherein the trans-splicing enhancer sequence consists of a chain of RNA nucleobases comprising at least one RNA motif having the formula X₁X₂X₃X₄X₅X₆, wherein:
X₁ is selected from the group consisting of adenine (A), uracil (U) and guanine (G);
X₂ is selected from the group consisting of adenine (A), uracil (U) and guanine (G);
X₃ is selected from the group consisting of adenine (A), uracil (U) and guanine (G);
X₄ is selected from the group consisting of adenine (A), uracil (U) and guanine (G);
X₅ is selected from the group adenine (A), uracil (U) and guanine (G); and
X₆ is selected from the group consisting of uracil (U) and guanine (G).

4. The composition of claim 1, wherein the trans-splicing enhancer sequence consists of a chain of RNA nucleobases comprising at least one RNA motif having the formula X₁X₂X₃X₄X₅X₆, wherein:
X₁ is selected from the group consisting of adenine (A), uracil (U) and guanine (G);
X₂ is selected from the group consisting of uracil (U) and guanine (G);
X₃ is selected from the group consisting of adenine (A), uracil (U) and guanine (G);
X₄ is selected from the group consisting of uracil (U) and guanine (G);
X₅ is selected from the group consisting of uracil (U) and guanine (G); and
X₆ is selected from the group consisting of uracil (U) and guanine (G).

5. The composition of claim 1, wherein the trans-splicing enhancer sequence is adjacent to RNA motifs that further increase trans-splicing efficiency.

6. The composition of claim 1, wherein the trans-splicing enhancer sequence is less than 300 bases from a 3′ splice site of the trans-splicing nucleic acid.

7. The composition of claim 1, wherein the trans-splicing enhancer sequence is less than 300 bases from a 5′ splice site of the trans-splicing nucleic acid.

8. The composition of claim 1, wherein each of the one or more intronic domains comprises 2 or more trans-splicing enhancer sequences.

9. The composition of claim 1, further comprising a 3′ untranslated region that increases trans-splicing efficiency.

10. The composition of claim 1, further comprising a 5′ untranslated region that increases trans-splicing efficiency.

11. The composition of claim 1, wherein the one or more replacement domains each comprises a gene expression-enhancing element.

12. The composition of claim 11, wherein the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response.

13. The composition of claim 1, further comprising an RNA-binding protein that strengthens the interaction between the trans-splicing nucleic acid and the target RNA molecule and increases trans-splicing efficiency.

14. The composition of claim 1, wherein the trans-splicing nucleic acid is RNA, DNA, a DNA/RNA hybrid, a nucleic acid analog, a chemically-modified nucleic acid, or a chimera composed of two or more nucleic acids or nucleic acid analogs.

15. The composition of claim 1, wherein the trans-splicing nucleic acid further comprises a heterologous promoter.

16. A vector comprising or encoding the composition of claim 1.

17. The vector of claim 16, wherein the vector is selected from the group consisting of: adeno-associated virus, retrovirus, lentivirus, adenovirus, nanoparticle, micelle, liposome, lipoplex, polymersome, polyplex, and dendrimer.

18. A cell comprising the vector of claim 16.

19. A method for treating a disease comprising administering to a patient in need of a therapeutically effective amount of the composition according to claim 1.

20. A method of correcting a genetic defect in a subject comprising administering to said subject the composition according to claim 1.
